(19) United States
(12) Patent Application Publication
(10) Pub. No.: US 2009/0142369 A1
(43) Pub. Date: Jun. 4, 2009

(54) Method for preventing skin-cellular injury by using green algae extract and cosmetic composition containing green algae extract

(76) Inventors: Meng-Han Shih, Taipei City (TW); Mei-Fen Shih, Taipei City (TW)

Correspondence Address: CHARLES E. BAXLEY, ESQUIRE, 90 JOHN STREET, SUITE 309, NEW YORK, NY, 10038, US

(21) Appl. No.: 11/998,499

(22) Filed: Nov. 30, 2007

(51) Int. Cl. A61K 36/02; C12N 9/99

(52) U.S. Cl. 424/195.17; 435/184

(57) ABSTRACT

A method for preventing UV-induced skin-cellular injury by using a green algae extract and a cosmetic composition containing the green algae extract are disclosed. An effective dose of the green algae extract is used to protect fibroblasts from UV-induced apoptosis. The mechanism of the disclosed method is using the green algae extract to inhibit interaction between apoptosis pathway molecules Fas/FasL so as to inhibit an adapter protein (FADD) in a death-inducing signaling complex (DISC) and in turn to inhibit caspase-3 activation and cleaved polymerase (PARP) such that irreparable damage to DNA can be prevented. The green algae extract can be mixed with a skin permeable cosmetic composition so that the cosmetic composition possesses the function of preventing skin from UV-induced injury.

### BACKGROUND OF THE INVENTION

1. Technical Field

The present invention relates to a method and a composition for preventing skin-cellular injury, which stems from excessive ultraviolet (UV) exposure. The present invention implements a green algae extract to prevent skin problems such as sunburn, irritation and cell death caused by UV-induced apoptosis in skin-cells.

2. Description of Related Art

It was introduced in 2002 by Matsumura and Ananthaswamy that UV exposure increases incidence of skin tumors because UV may incur DNA damage, error genetic information as well as cell proliferation signals and further lead to extracellular epigenetic effects.

Also, Dunkem et al addressed the theory in 2001 that UV-induced apoptosis in fibroblasts has been demonstrated to be mediated through cysteine-aspartic acid-specific-protease (caspase-3) activation subsequently irreparable DNA damage due to polymerases (PARP) cleavage.

Apoptosis mentioned in the above theories is a death process carried out orderly as a natural response of cells to physiologic or pathologic stimulus, environmental changes or progressive damages. The process primarily includes the following steps:
(I) Apoptosis Signal Transmission: After inductive factors act on cells, apoptosis signals are transmitted into the cells through complex transmission pathways and the cells accordingly decide to survive or die;
(II) Apoptosis Gene Activation: Apoptosis signals promote apoptosis genes;
(III) Apoptosis Execution: In the cells deciding to die, apoptosis is actuated according to a preset program and then enzymes as well as substances related to biodegradation are activated, resulting in formation of apoptosomes; and
(IV) Elimination of Dead Apoptotic Cells: The dead apoptotic cells are engulfed by adjacent phagocytes and degraded in those phagocytes.

From a viewpoint of morphology, a series of cellular changes during the apoptotic progress primarily comprises: membrane blebbing, membrane buding, cytoplasm dehydration, irregular morphology caused by cellular shrinkage, intracellular mitochondria swell and vacuolation, chromatin concentrating to chromatin mass and chromatin margination or centronation, and chromatin mass broken into fragments. From a viewpoint of biochemistry, cellular changes during the apoptotic progress primarily comprise: DNA degradation, endogenous activation of endonucleases, and activation of caspases.

Caspases mentioned in the above theories are substantively enzymes and 14 types of caspases have been found to date. The 14 types of caspases may be classified into three groups according to the structures and functions thereof. The first group includes caspase-2,-8,-9,-10, which have large-prodomain and are initiator proteases of apoptosis. The second group includes caspase-3,-6,-7, which have small-prodomain and closely relates to the final execution of apoptosis. The third group includes caspase-1,-4,-5,-14, which mainly relates to the generation of inflammatory signals as well as immunoreaction and has less connection with apoptosis.

In a word, caspases play an important role in the actuation and completion of apoptosis. Once caspases are activated, cells are led to death. Caspase activation not linking to mitochondrion involves the practice that when an exterior death ligand combines a death recepter on the cell membrane, precursors of caspases will be activated to generate active caspases and then more caspases will be activated through a series of caspase cascades so as to cause apoptosis.

It has been found through recent researches that caspase activation not linking to mitochondrion is based on the interaction between apoptosis pathway molecules Fas/FasL. Therein, Fas is a type 1 membrane protein and also referred to as APO-1 or CD95. It can form homotrimers that take charge of transmitting apoptosis information to the inside of cells. The ligand of Fas is FasL (Fas Ligand), also referred to as APO-1 or CD95L. It is a type 2 membrane protein. As currently known, activated FasL exists as homotrimer molecules. When FasL combines with Fas on the surfaces of cell membranes, the apoptosis pathway is activated. After Fas is triggered by FasL, plural proteins gather at a death domain of Fas to form a death-inducing signaling complex (DISC). In the death-inducing signaling complex (DISC), one adapter protein, Fas-associated death domain (FADD), also referred to as Mort1, acts as a bridge between Fas and the precursor of the caspases, namely procaspase 8, so as to result in oligomerization of caspase-8 and in turn to activate the downstream caspase-3. The activated caspases can destroy various proteins, such as cytoskeleton Actin Gas 2, Lamins, polymerases (PARP) taking charge of DNA reparation, DNA-PK and signal transduction protein PKC8.

### SUMMARY OF THE INVENTION

It has been proven by many researches that excessive ultraviolet (UV) exposure may cause skin-cellular injury and incur symptoms, such as sunburn and irritation, and eventually lead to cell death (apoptosis). It is the main objective of the present invention to provide a method to prevent UV-induced apoptosis in skin-cells and a skin permeable formulation employing the method.

To achieve these and other objectives of the present invention, the present invention employs an effective dose of a green algae extract to protect skin-cells from premature apoptosis induced by excessive UV exposure. The mechanism of the disclosed method is using the green algae extract to inhibit interaction between apoptosis pathway molecules Fas/FasL so as to inhibit an adapter protein (FADD) in a death-inducing signaling complex (DISC) and in turn to inhibit caspase-3 activation and cleaved polymerase (PARP) such that irreparable damage to DNA can be prevented. The green algae extract can be mixed with a skin permeable cosmetic composition so that the cosmetic composition possesses the function of preventing UV-induced skin injury.

The method for preventing UV-induced skin-cellular injury uses the green algae extract to inhibit the caspase activity induced by UV so to inhibit apoptosis in human skin fibroblasts.

The method for preventing UV-induced skin-cellular injury uses the green algae extract to inhibit the adapter protein, FADD, produced in UV-induced interaction between apoptosis pathway molecules Fas/FasL, from being phosphorylated and in turn being activated.

The method for preventing UV-induced skin-cellular injury uses the green algae extract to inhibit UV-induced PARP cleavage.

A preferable application of the present invention is to integrate an effective dose of the green algae extract into a cosmetic composition for human skin so as to endow the composition with ability to prevent skin-cellular injury.

The cosmetic composition integrated with the green algae extract of the present invention is ideal for protecting skin and preventing UV-induced apoptosis as well as UV-induced skin problems such as sunburn and irritation.

The actual dose of the green algae extract used in the present invention is not definitely limited and may vary in different preparation methods.

The green algae extract and the skin permeable composition integrated with the green algae extract of the present invention may be provided in any form, such as aqueous liquid, gel, lotion, cream, paste, powder and oily liquid. A preferred range for the green algae extract is in the range of about 0.01 to about 50% by weight of the total of composition.

### BRIEF DESCRIPTION OF THE DRAWINGS

The invention as well as a preferred mode of use, further objectives and advantages thereof, will best be understood by reference to the following detailed description of an illustrative embodiment when read in conjunction with the accompanying drawings, wherein:

FIG. 1 is a histogram, showing the protective effects of the green algae extract of the present invention on UV-induced cytotoxicity;

FIG. 2 is a histogram, showing the inhibitory effects of the green algae extract of the present invention on UV-induced caspase 3 activity;

FIG. 3 shows the inhibitory effects of the green algae extract of the present invention on UV-induced PO4-FADD analyzed by the western blotting method;

FIG. 4 shows the inhibitory effects of the green algae extract of the present invention on UV-induced PARP cleavage analyzed by the western blotting method;

FIG. 5 is an IR spectrum of the green algae extract of the present invention;

FIG. 6 is another IR spectrum of the green algae extract of the present invention; and

FIG. 7 is still another IR spectrum of the green algae extract of the present invention.

### DETAILED DESCRIPTION OF THE PREFERRED EMBODIMENT

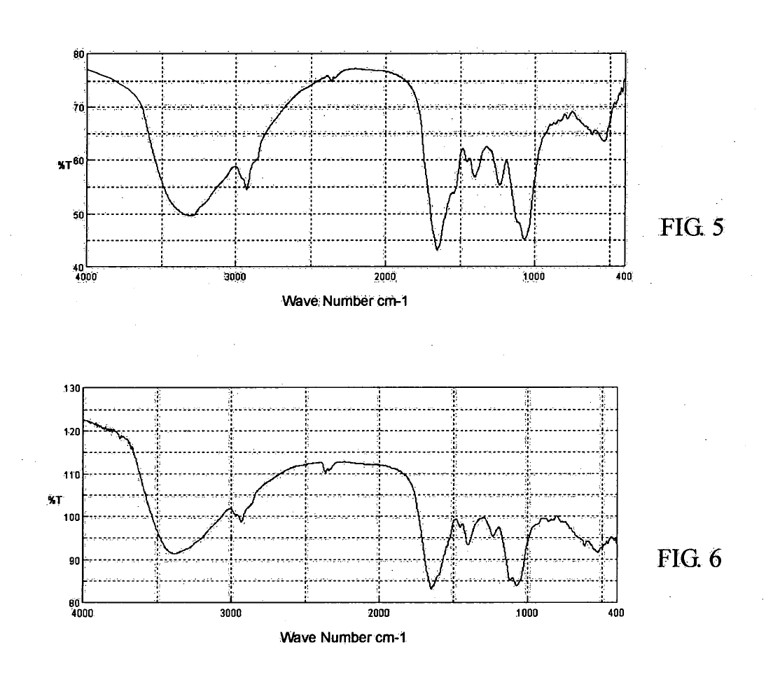

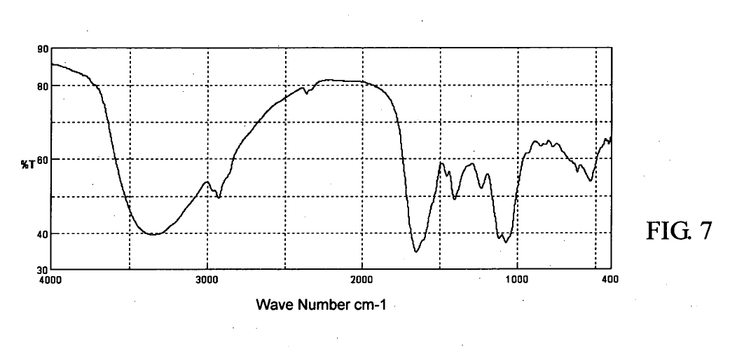

The present invention discloses a method for preventing UV-induced skin-cellular injury by providing a green algae extract as a skin-cell protector to inhibit interaction between apoptosis pathway molecules Fas/FasL so as to inhibit caspase-3 activation and polymerase (PARP) cleavage, such that unrecoverable damage to DNA can be prevented. The green algae extract may be made through any commercially available method and mainly achieved by extracting an aqueous liquid extract from fresh water grown unicellular algae. From the IR spectrums of FIGS. 5, 6 and 7, wherein the wave numbers are ranging form 4000 cm-1-400 cm-1, it is learned that the green algae extract is substantially an organic compound and the major elements therein are carbon, hydrogen, as well as nitrogen. The concentrations of the elements are: nitrogen (N) 7.06%˜7.66%, hydrogen (H) 5.9%˜6.51%, and carbon (C) 35.67%˜38.1%.

For demonstrating the protective effects of the green algae extract on UV-induced skin injury, the results of some experiments are provided for further illustration.

### [Experiment 1]

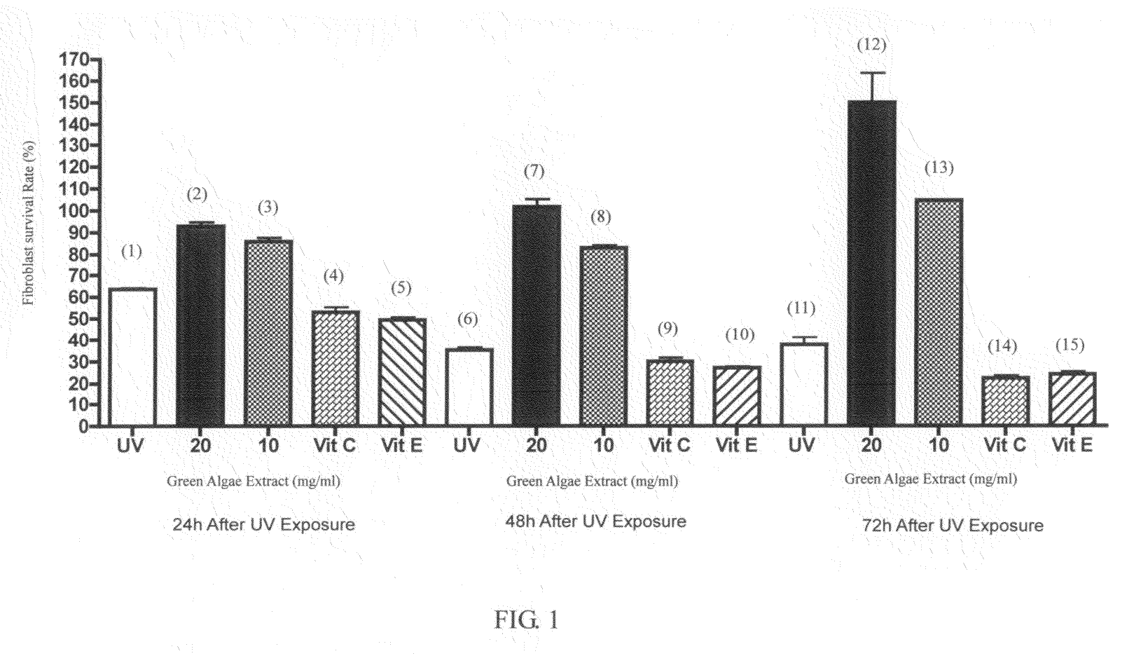

FIG. 1 shows the results of experiment (n≧6) that demonstrates the protective effects of the green algae extract of the present invention on UV-induced cytotoxicity, namely the ability of the green algae extract to prevent UV-induced skin-cellular injury. Green algae extracts (20 mg/ml and 10 mg/ml), Vitamin C, and Vitamin E treated fibroblasts are taken as subjects of the present experiment. The fibroblast survival rates are analyzed after 24, 48 and 72 hours of UV exposure and the results are quantified in the histogram.

In the histogram, Rectangles (1), (6) and (11) belong to a comparison group, and the resultant data exhibit that the fibroblast survival rates of the fibroblasts after 24, 48 and 72 hours of UV exposure are respectively about 63%, 35%, and 37% of the fibroblast survival rates of a control group (100%) which is not exposed in UV. Thus, it is revealed that UV exposure is responsible for the decreased fibroblast survival rates and thus the fact is proven that UV-induced cytotoxicity significantly affects the fibroblast survival rates.

In the histogram, Rectangles (2), (7) and (12) belong to an experimental group, and the resultant data exhibit that the fibroblast survival rates in the green algae extract of 20 mg/ml after 24, 48 and 72 hours of UV exposure are respectively about 92%, 101%, and 149% of the fibroblast survival rates of the control group which is not exposed in W. As compared with the comparison group, the fibroblast survival rates of the experimental group are much higher. Thus, the fact is proven that an effective dose of the green algae extract facilitates reducing UV-induced cytotoxicity so as to provide protection to cells and in turn increase the cell survival rates.

In the histogram, Rectangles (3), (8) and (13) belong to another experimental group, and the resultant data exhibit that the fibroblast survival rates in the green algae extract of 10 mg/ml after 24, 48 and 72 hours of UV exposure are respectively about 85%, 82%, and 104% of the fibroblast survival rates of the control group which is not exposed in UV. As compared with the comparison group, the fibroblast survival rates of the experimental group are higher. Thus, the fact is proven that an effective dose of the green algae extract facilitates reducing UV-induced cytotoxicity so as to provide protection to cells and in turn increase the cell survival rates.

In the histogram, Rectangles (4), (9) and (14) belong to another experimental group, and the resultant data exhibit that the fibroblast survival rates in Vitamin C of 0.15 mM after 24, 48 and 72 hours of UV exposure are respectively about 53%, 29%, and 22% of the fibroblast survival rates of the control group which is not exposed in U. As compared with the comparison group, the present experimental group fails to sustain fibroblast survival rates. Thus, the fact is proven that Vitamin C does not facilitate reducing UV-induced cytotoxicity and fails to provide protection to cells.

In the histogram, Rectangles (5), (10) and (15) belong to another experimental group, and the resultant data exhibit that the fibroblast survival rates in Vitamin E of 25 μM after 24, 48 and 72 hours of UV exposure are respectively about 49%, 27%, and 23% of the fibroblast survival rates of the control group which is not exposed in U. As compared with the comparison group, the present experimental group fails to sustain fibroblast survival rates. Thus, the fact is proven that Vitamin E does not facilitate reducing UV-induced cytotoxicity and fails to provide protection to cells.

### [Experiment 2]

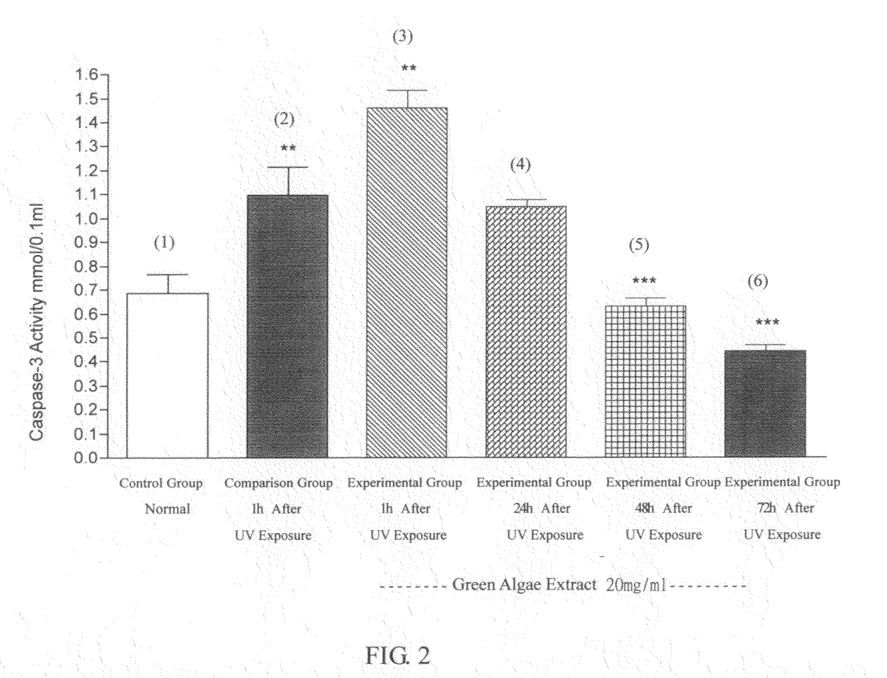

As mentioned previously, caspases play an important role in the actuation and completion of apoptosis. Thus, the present experiment activates caspase-3 by UV to analyze the inhibitory effects of the green algae extract of the present invention on caspase-3 activity. The green algae extract of 20 mg/ml containing fibroblasts is taken as a subject of the present experiment. The concentrations of caspase-3 are tested after 1, 24, 48 and 72 hours of UV exposure and the results are quantified in the histogram of FIG. 2.

In the histogram, Rectangle (1) belongs to a non-UX exposed control group and exhibits that the caspase-3 concentration in fibroblasts is normally 0.7 mmol/0.1 ml. Rectangle (2) belongs to a comparison group and exhibits that after one hour of UV exposure, caspase-3 in fibroblasts is activated and the concentration thereof is increased to about 1.1 mmol/0.1 ml. This result proves that UV exposure substantially induces caspase-3 activation.

In the histogram, Rectangle (3) belongs to an experimental group and exhibits that the caspase-3 concentration in fibroblasts in green algae extract of 20 mg/ml after one hour of UV exposure is increased to about 1.4 mmol/0.1 ml. This result proves that caspase-3 currently has been activated in a higher rate. This early activation of apoptotic enzyme may indicate that green algae extract accelerates UV-induced cell death in order to maintain normal cell population which is evidence in cell survival rate in experiment 1. In the histogram, Rectangle (4) belongs to an experimental group and exhibits that the caspase-3 activity in fibroblasts in the green algae extract of 20 mg/ml after 24 hours of UV exposure is reduced to about 1.1 mmol/0.1 ml from about 1.4 mmol/0.1 ml that measured after one-hour UV exposure. Inhibition of caspase-3 is kept proceeding. As shown by Rectangles (5) and (6), after 48 hours and 72 hours of UV exposure, the green algae extract of the present invention still works on restraining caspase-3 activity, and the caspase-3 concentrations is reduced to respectively 0.6 mmol/0.1 ml and 0.4 mmol/0.1 ml.

Experiment 2 thus proves that an effective dose of the green algae extract of the present invention performs evident effect on inhibiting caspase-3 so as to protect fibroblasts from premature apoptosis.

### [Experiment 3]

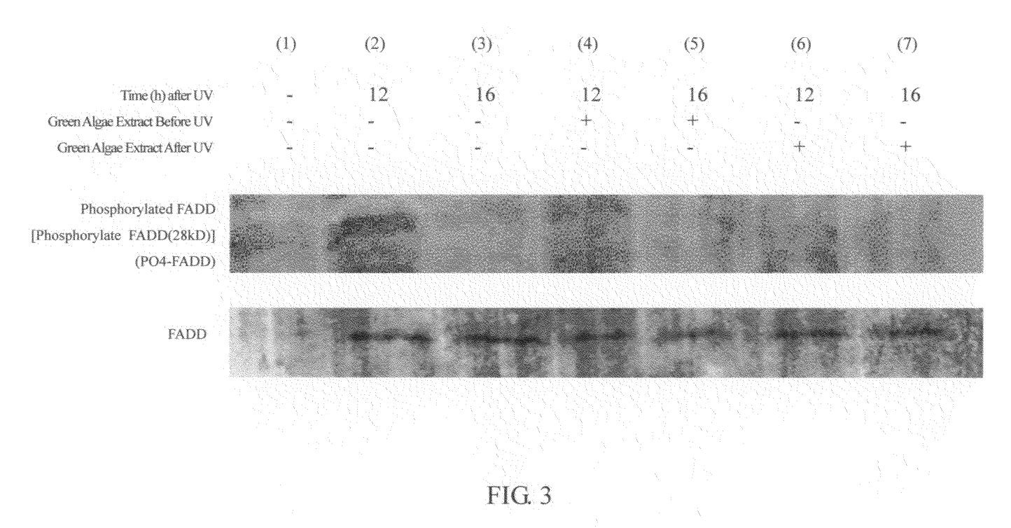

It is known that caspase activation is achieved by the death-inducing signaling complex (DISC) which is a product of the interaction between apoptosis pathway molecules Fas/FasL. As described above, Fas-associated death domain (FADD) is one of the death-inducing signaling complex (DISC) and phosphorylated FADD is in the form of an activated FADD protein. Only such phosphorylated FADD can act as the bridge between Fas and procaspase 8, which is the precursor of the caspase, and lead to oligomerization of caspase-8 to activate the downstream caspase-3. Thereupon, the present experiment activates FADD by UV or phosphorylates FADD, to form Phosphorylated FADD (PO4-FADD), to analyze the inhibitory effects of the green algae extract of the present invention on PO4-FADD by the western blotting method. The results are shown in FIG. 3.

In FIG. 3, Column (1) belongs to a control group and exhibits the FADD and PO4-FADD expressions in fibroblasts under basal condition.

Columns (2) and (3) belong to a comparison group, and exhibit FADD and Po-4 FADD expressions at the 12^{th} hour and 16^{th} hour after UV exposure. As compared with the control group shown by Column (1), FADD and PO4-FADD of the comparison group are increased.

Columns (4) and (5) belong to an experimental group wherein the green algae extract of 20 mg/ml is added in fibroblasts before the fibroblasts are exposed to UV irradiation. FADD and PO-4. FADD expressions are observed at the 12^{th} hour and 16^{th} hour after UV exposure. As compared with the comparison group shown by Rectangles (2) and (3), PO4-FADD of the experimental group are decreased.

Columns (6) and (7) belong to another experimental group wherein the green algae extract of 20 mg/ml is added in fibroblasts that have be exposed in UV. FADD and PO-4 FADD expressions are observed at the 12^{th} hour and 16^{th} hour after UV exposure. As compared with the comparison group shown by Columns (2) and (3), PO4-FADD of the experimental group are decreased.

Experiment 3 proves the fact that the effective dose of the green algae extract added in fibroblasts, no matter before or after UV exposure, facilitates inhibiting PO4-FADD expressions so as to reduce the bridges between apoptosis pathway molecule Fas and procaspase 8, the precursor of the caspase, and in turn reduce oligomerization of caspase-8, resulting in reduction of caspase-3. Consequently, UV-induced premature apoptosis in fibroblasts is prevented.

### [Experiment 4]

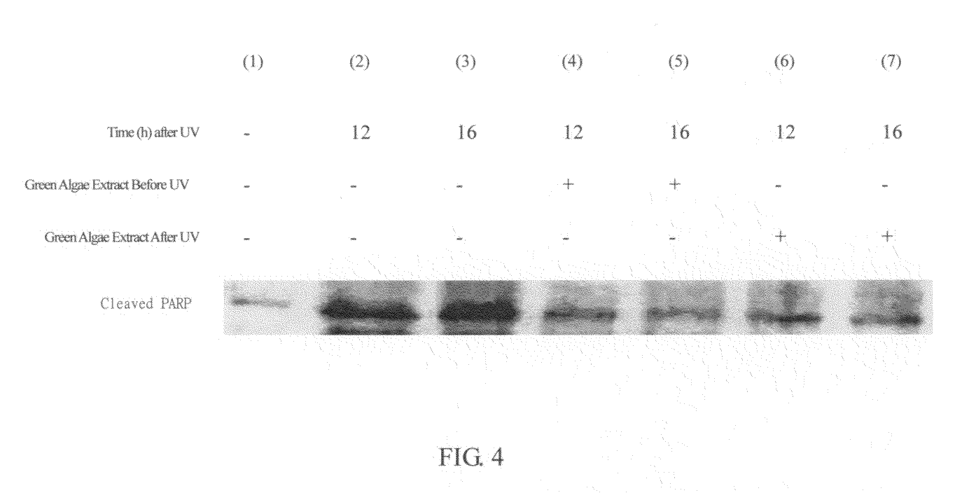

It is known that UV-induced fibroblast apoptosis stems to caspase activation and causes polymerase (PARP) cleavage that renders irreparable damage to DNA. Experiments 1, 2 and 3 have proven that the green algae extract of the present invention facilitates inhibiting PO4-FADD and caspase-3 activities so that PARP cleavage is expectably reduced. Thereupon, the present experiment uses UV to cause cleaved PARP and analyzes the inhibitory effects of the green algae extract of the present invention on cleaved PARP by the western blotting method. The results are shown in FIG. 4.

In FIG. 4, Column (1) belongs to a control group and exhibits cleaved PARP expression in fibroblasts under basal condition.

Columns (2) and (3) belong to a comparison group, and exhibit cleaved PARP expressions at the 12^{th} hour and 16^{th} hour after UV exposure. As compared with the control group shown by Column (1), both cases show increased amounts of cleaved PARP.

Columns (4) and (5) belong to an experimental group wherein the green algae extract of 20 mg/ml is added in fibroblasts before the fibroblasts are exposed in V. The expression of cleaved PARP is observed at the 12^{th} hour and the 16^{th} hour after UV exposure. As compared with the comparison group shown by Columns (2) and (3), the amounts of cleaved PARP in the present experimental group are decreased.

Columns (6) and (7) belong to another experimental group wherein the green algae extract of 20 mg/ml is added into fibroblasts that have been exposed in UV. Then the expression of cleaved PARP is observed at the 12^{th} hour and 16^{th} hour. As compared with the comparison group shown by Columns (2) and (3), the amounts of cleaved PARP in the present experimental group are decreased.

Experiment 4 therefore proves the fact that the effective dose of the green algae extract added in fibroblasts, no matter before or after UV exposure, facilitates inhibiting the expression of PARP cleavage so as to protect fibroblasts from UV-induced premature apoptosis.

The aforementioned experiments are for purposes of illustration and demonstration and not limitations to the practice of the present invention.

What is claimed is:

1. A method for preventing UV-induced skin-cellular injury by using a green algae extract, comprising:
employing an effective dose of the green algae extract to inhibit a UV-induced activity of the caspase in a fibroblast and to inhibit an apoptosis in the fibroblast.

2. The method of claim 1, wherein the caspase is a cysteine-aspartic acid-specific-protease, also referred to as caspase-3.

3. The method of claim 2, further comprising employing the effective dose of the green algae extract to inhibit an expression of a phosphorylated FADD (PO4-FADD) formed by a UV-induced interaction between apoptosis pathway molecules Fas/FasL in the fibroblasts.

4. The method of claim 2, further comprising employing the effective dose of the green algae extract to inhibit a UV-induced cleavage of a polymerase (PARP) in the fibroblast.

5. A cosmetic composition for preventing UV-induced skin-cellular injury by using a green algae extract, providing an effective dose of the green algae extract in a skin permeable cosmetic composition so as to inhibit a UV-induced activity of a caspase in a fibroblast and to inhibit an apoptosis in the fibroblast.

6. The cosmetic composition of claim 5, wherein the green algae extract is an aqueous liquid extract extracted from water grown unicellular algae.

7. The cosmetic composition of claim 5, wherein the cosmetic composition is in a form selected from the group consisting of aqueous liquid, gel, lotion, cream, paste, powder and oily liquid.

8. The cosmetic composition of claim 5, wherein the green algae extract is in a range of about 0.01 to about 50% by weight of the cosmetics composition.

9. A method for preventing UV-induced skin-cellular injury by using a green algae extract, comprising mixing an effective dose of the green algae extract in a skin permeable cosmetic composition so as to inhibit a UV-induced activity of a caspase in a fibroblast.
